(19) United States
(12) Patent Application Publication
(10) Pub. No.: US 2006/0008471 A1
(43) Pub. Date: Jan. 12, 2006

(54) Organ, tissue and cell-specific immuno-therapeutic for chronic viral infections and inflammatory, degenerative and proliferative diseases, in particular of the liver, and for cancer, based on a recombinant parapox virus

(75) Inventors: Olaf Weber, Wulfrath (DE); Angela Siegling, Paris (FR); Tobias Schlapp, Koln (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Aktiengesellschaft, Leverkusen (DE)

(21) Appl. No.: 11/225,508

(22) Filed: Sep. 13, 2005

Related U.S. Application Data
(63) Continuation of application No. 10/009,856, filed on Apr. 10, 2002.

(30) Foreign Application Priority Data
May 14, 1999 (DE) 199 22 407.2

(51) Int. Cl. A61K 39/12

(52) U.S. Cl. 424/199.1

(57) ABSTRACT

The present invention relates to the preparation and use of organ-specific, tissue-specific and/or cell-specific recombinant parapoxvirus ovis as a pathogen-specific and organ-specific, targeted immunotherapeutic agent for chronic viral infections and inflammatory, degenerative and proliferative diseases, in particular of the liver, and cancer.

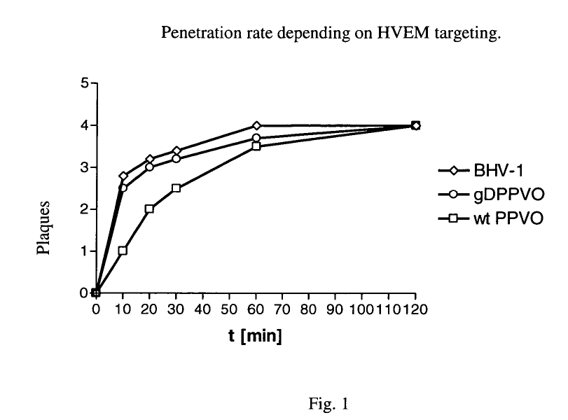

The present invention relates to the preparation and use of recombinant parapoxvirus for the organ-specifically, tissue-specifically and/or cell-specifically targeted immunotherapy of viral infections and inflammatory, degenerative and proliferative diseases, particularly of the liver, and cancer. It furthermore relates to the use of recombinant parapoxvirus possessing targeting properties for producing pharmaceuticals.

Diseases of the skin and its adnexa, of the internal organs, of the central nervous system and its adnexa, including the eye, and also cancer, in both humans and animals, also come within the area of application of the abovementioned parapoxviruses.

It is known that latent and chronically persistent viral infections can be activated or reactivated by immunosuppression or, conversely, that the immune system suppresses the acute disease which can be induced by a virus which is latent (e.g. a latent herpesvirus infection recurs in association with immunosuppression: blisters on the lips in association with stress or cortisone administration). It is furthermore known that chronically persistent and latent viral infections are difficult to treat, or cannot be treated at all, with conventional antiviral substances which are based on low molecular weight compounds.

A reason for this may be that such infections are associated with the lack of a viral enzymic activity (for example the lack of a viral polymerase activity which has first of all to incorporate a nucleosidic inhibitor into the viral nucleic acid so that this inhibitor can then, for example, bring about chain breakage in the viral DNA; for example, the lack of a viral thymidine kinase activity which, for example, has first of all to phosphorylate an antiviral compound so that this compound can become active) or else that the immune system of the host fails to recognize infected cells or viral antigens.

It is likewise known that, in the case of chronically persisting viral infections, superinfection with another virus can lead to antiviral effects which are directed against the chronically persisting virus¹⁾. The authors have been able to demonstrate that this effect is dependent on interferons (in particular IFN-γ) and TNF-α, which are secreted by T cells, natural killer cells and macrophages.

The results obtained by these authors confirmed another, earlier study which showed that class I-restricted cytotoxic T cells were able to inhibit hepatocellular HBV gene expression in HBV-transgenic mice, that this process took place without the liver cells being destroyed and that the process was elicited by TNF-α and IFN-γ²⁾.

A product for inducing “paraspecific immunity”, i.e. what is termed a paraimmunity inducer, has been used both therapeutically and metaprophylactically and prophylactically in veterinary practice for a relatively long time. These paraimmunity inducers consist, for example, of chemically inactivated parapoxvirus ovis. BAYPAMUN® (DE 3504940) is a product which is produced on the basis of this virus (parapoxvirus ovis, strain D 1701).

In animals, the inactivated virus induces nonspecific protection against infections by a very wide variety of pathogens. It is assumed that the animal's endogenous defense system mediates this protection by way of a variety of mechanisms.

These mechanisms include: induction of interferons, activation of the natural killer cells, induction of “colony-stimulating activity” (CSA), and stimulation of lymphocyte proliferation. Earlier investigations on the mechanism of action demonstrated stimulation by interleukin 2 and interferon-γ³⁾.

It is likewise known that parapoxviruses can be provided, as vectors, with genes from other pathogens in order to be able to express the corresponding proteins and thus generate prophylactic immunoprotection (vaccination) against the donor pathogen⁴⁾.

It is furthermore known that recombinant, so-called “pseudotyped” viruses are able to infect target cells, tissues, organs and/or hosts which it was not originally possible to infect⁵⁾.

The possibility of using vectors in targeted gene therapy on the basis of these findings has already been discussed⁶⁾.

In pharmacology, use is made of natural and synthetic molecules, such as asialofetuin or poly-L-lysine in order to make particular organs, in the case of the examples mentioned here, the liver, selectively available for therapy with these molecules on the basis of interaction with organ-specific receptors, in the case of the examples mentioned here, the asialoglycoprotein receptor of the liver⁷⁾.

Against this background, the object therefore arises of further improving the therapeutic utility of the outstanding immunogenic effect of parapoxvirus ovis such that the above-described, generalized paraspecific immunogenicity of the parapoxviruses can be directed in a targeted manner toward the diseased organ (system) and the causative pathogen.

Focusing in this way would make it possible to expect a therapeutic effect which would be associated with fewer side-effects and which would be expressed more powerfully and more persistently at the site of action.

The object of the invention was therefore to generate the immunological effect of the parapoxvirus in a targeted manner. The object is achieved by coupling or introducing suitable foreign peptides or proteins, which are able to interact with organ-specific, tissue-specific and/or cell-specific receptor molecules, to or, respectively, into the virus.

In this way, we were able to powerfully focus the immune reaction. This thereby makes it possible, for the first time, to use parapoxvirus ovis to concentrate the complex capacity of the immune system at the site where it is required.

The advantages which ensue from this consist in tissue specificity, organ specificity or cell specificity which is associated with a concomitant reinforcement of the immunological effect at the site at which it is required, and in a decrease in side-effects.

Since undesirable side-effects of a general nature have, on the one hand, to be expected, and/or, on the other hand, only an insufficient concentration of the active compound is achieved at the site of action, when the previously known methods/products are applied systemically, it is possible to use the novel type of parapoxvirus ovis which is described here to achieve a therapy which is more target specific and more effective.

In order to prepare recombinant parapoxvirus ovis for targeted organ-specific, tissue-specific and/or cell-specific immunotherapy, it is possible to use known viral proteins/peptides which can be either unmodified or modified, or elongated or truncated. In this connection, the large envelope protein of the human hepatitis B virus (HBV) has, for example, proved to be particularly suitable for reaching the liver.

In addition, it is possible to use nonviral proteins/peptides, in particular asialoglycoprotein, for the targeted therapy of the liver.

It is also possible to use novel synthetic proteins/peptides whose sequences can be identified, for example from phage libraries, using techniques with which the skilled person is familiar⁸⁾.

In addition to the peptides or proteins which have been mentioned, it is also possible to clone immunomodulatory epitopes, which have been selected, for example, from hepatitis B virus or other viruses, or tumor-associated antigens, into the parapoxvirus.

In this way, an immunostimulatory property, which is directed powerfully and specifically against the pathogen or the tumor, is introduced into the parapoxvirus.

Suitable epitopes are identified using known techniques with which the skilled person is familiar, for example flow cytometry⁹⁾.

Novel recombinant viruses possessing the above-described properties can, for example, be prepared and characterized as described below:

Preparation of a recombinant virus which lacks sequences whose gene products, or parts thereof, are not required for the immunomodulatory effect or for viral replication.

An example of the cloning of the recombinant parapoxvirus ovis takes, as its starting point, the construction of double selection cassettes, which express one marker gene, for example the LacZ gene under the control of the Vaccinia 11K gene or of another suitable sequence, and another selection marker gene, for example the gpt gene (encodes the enzyme xanthine-guanine phosphoribosyltransferase, XGPRT) under the control of the correspondingly suitable promoter. The viral sequences can then, for example, be deleted as described below:

Unique restriction cleavage sites in a region of parapoxvirus ovis which is not essential either for viral replication or for the immunomodulatory effect, for example a suitable envelope protein gene, another gene which encodes a structural protein (subsequently termed a suitable gene), or another gene, for example the VEGF gene, are used as starting points for bringing about the bidirectional deletion of sequences under the influence of the endonuclease Bal31.

For this, the corresponding plasmid, for example, a suitable structural protein gene which contains the parapoxvirus ovis nucleic acid sequence, is opened in the VEGF gene using a suitable restriction enzyme, and the plasmid, which has now been linearized, is incubated with Bal31. Suitable deletion plasmids are filled in and oligonucleotides which are complementary thereto, and which constitute new unique cleavage sites, for example SmaI, SalI and EcoRV restriction cleavage sites, are ligated to the Bal31 products, which have been provided with smooth ends.

After the transformation of bacteria, the plasmid DNA can be isolated and cleaved with an enzyme which contains no recognition site in the sequence of the corresponding parapoxvirus ovis DNA fragment. After the LacZ/gpt selection cassette, which has been cleaved with the corresponding restriction enzymes, has been inserted into the deletion site in the suitable gene, the precise size of the deletions which have been produced in each resulting recombinant plasmid DNA can be determined by sequencing.

The virus, which then lacks the corresponding gene product, or a part thereof, can, for example, be prepared as follows:
Suitable cells, such as bovine kidney cells, which have grown to confluence are infected with an infective dose of approx. 0.1 multiplicity of infection (moi). After about 2 hours, the infected cells are transfected, for example using transfection systems with which the skilled person is familiar and which are commercially available, with a deletion plasmid (e.g. 10 μg) which has been prepared as described above. Subsequently, these cell cultures are incubated, at approximately 37° C. for 3 to 6 days and in an approximately 5% CO2 atmosphere, with a suitable selection medium (e.g. with HAT medium [hypoxanthine-aminopterin-thymidine], MPA [mycophenolic acid]) until a cytopathic effect (cpe) or plaque formation is visible. The cells are then lysed, after which a dilution series is prepared from the cell lysate and a plaque test is carried out on suitable cells. For the plaque test, an agarose medium mixture, which can contain, for example, approximately 0.3 mg of Bluo-Gal (GIBCO)/ml, is added in order to identify blue plaques, which, for example, contain LacZ-expressing, MPA-resistant recombinant viruses. The recombinant viruses which have been obtained in this way are used for infecting suitable cells, such as bovine kidney cells, and are subjected to at least two further plaque titrations until a recombinant virus population which is as homogeneous as possible, and which is most advantageously >99.9% homogeneous, has been obtained.

Preparing a recombinant virus which contains sequences whose gene products, or parts thereof, are required for organ-specific, tissue-specific or cell-specific targeting.

An analogous approach is used for preparing the recombinant virus containing targeting sequences. A virus which has been altered as described above is used as the starting virus. Alternatively, the targeting sequence can be incorporated into a virus which has not been genetically altered if this does not have a negative influence on virus replication and/or the immunomodulatory effect. Instead of the plasmid which contains deleted or truncated sequences of parapoxvirus ovis, use is made of a corresponding plasmid which contains a DNA sequence which is unaltered, or is altered in a suitable manner, and which encodes a protein or peptide which enables the recombinant virus, in non-inactivated form or in inactivated form, to be targeted in an organ-specific, tissue-specific and/or cell-specific manner. If it is desired, for example, to introduce the recombinant virus into the liver, this sequence can, for example, be the sequence for the large envelope protein of human hepatitis B virus, or another suitable sequence. If the targeting sequence is incorporated into a gene which does not encode a structural protein, the targeting sequence can then be coupled to appropriate membrane anchors in order to enable it to be incorporated into the virus envelope.

The choice of the selection markers in connection with the preparation is to be made such that there is no interference, or only suitable interference, with selection markers which are already present.

In an analogous manner, it is possible, in addition, to insert sequences which encode immunologically active epitopes. These epitopes can be selected using methods which are known to the skilled person⁹⁾.

Detecting the Targeting Properties of the Recombinant Virus.

The new properties of the recombinant virus are detected, on the one hand, in the case of this virus, using suitable methods which are known to the skilled person, such as the use of selection markers and/or detection of the new protein/peptide by means of Western blotting; on the other hand, it is possible to carry out a functional detection. This latter is performed on cells which are being targeted. When the liver is being targeted with a recombinant virus which contains asialoglycoprotein or appropriate parts thereof, this functional detection can be performed by detecting the binding of recombinant virus to cells which are expressing the asialoglycoprotein receptacle. These cells can be human liver cells or hepatoma cells (e.g. HepG2) in which it is possible to carry out competitive binding studies using asialoglycoprotein and recombinant virus.

As a control, these studies are also performed on cells which are not expressing the asialoglycoprotein receptor, for example fibroblasts. The targeting properties are present both in inactivated recombinant viruses and in recombinant viruses which are not inactivated. However, for therapy purposes, use is only made of those recombinant viruses whose targeting properties have been demonstrated to correspond to the therapeutic objective.

Detecting the Immunomodulatory Properties

The immunomodulatory properties of the recombinant virus can be detected experimentally in mice, for example. For this, the recombinant virus, in inactivated or non-inactivated form, is injected, for example into a body cavity, for example intraperitoneally or subcutaneously, intramuscularly or intravenously, in mice, for example Balb/c mice. In accordance with a schedule which is to be established, for example 6, 12 and 24 hours after the administration, the animals are sacrificed and organs and/or cells, for example cells which are obtained by peritoneal lavage, are removed. Genetic material, such as RNA, is isolated from the organs/cells, and the expression of cytokines is determined using suitable methods, for example by means of semiquantitative or quantitative PCR.

For a particular therapy, use is then made of those recombinant viruses whose immunomodulatory properties (induction of a Th1 immune response) suggest that a therapeutic effect is to be expected.

On the basis of the known circumstances of the influence of a Th1 immune response on latent and chronically persistent viral infections^{10,11)} and the immunomodulatory properties of the recombinant parapoxvirus ovis, which properties are similar or superior to those of non-recombinant parapoxvirus ovis, it is possible to use organ-specific, tissue-specific and/or cell-specific recombinant parapoxvirus ovis as a monotherapy, or in combination with biologically active (e.g. antiviral), low molecular weight compounds or biologically active proteins, in humans and animals, with this use being of therapeutic value for the antiviral therapy of mainly chronic infections with hepatitis B virus, or other viral infections of the internal organs, especially the liver, where mention may be made, by way of example, of hepatitis C virus (HCV) or of all the other pathogens from the group of hepatitis-causing viruses¹²⁾, and infections, also when accompanied by other diseases, with the various types of herpes simplex virus (HSV), the various types of human papilloma virus (HPV), human immunodeficiency virus (HIV) and human cytomegalovirus (HCMV), and also the corresponding viral diseases in animals.

Furthermore, on the basis of the mechanism of action which has been indicated, it is possible to use the recombinant parapoxvirus for carrying out the following prophylactic or therapeutic treatments, in particular, with some prospect of achieving success:

Preventing recurrences in connection with herpesvirus infections, and metaphylaxis, i.e. the prevention of the establishment of viral infections (e.g. HIV) when the patient is treated with the agent immediately following exposure¹³⁾. Based on the mechanism of action, it is likewise possible to treat cancer^{14,15)}. It is possible to use organ-specific, tissue-specific and/or cell-specific recombinant parapoxvirus ovis strains as a monotherapy, or in appropriate combination with biologically active, low molecular weight compounds, in the said indications as well.

It is likewise possible to use recombinant parapoxvirus ovis to treat inflammatory and non-inflammatory degenerative and proliferative diseases of the liver such as liver cirrhosis and/or liver fibrosis. It is possible to use organ-specific, tissue-specific and/or cell-specific recombinant parapoxvirus ovis strains as a monotherapy or in appropriate combination with biologically active, low molecular weight compounds in connection with these said indications as well.

Recombinant virus is prepared for organ-specific, tissue-specific and/or cell-specific therapy depending on the clinical problem (for example chronic hepatitis B virus disease in humans).

The procedure is to delete or mutate genes which are not required for inducing a cell-mediated immune response. The gene sequences encoding epitopes (peptide/proteins) which ensure specific interaction with one or more receptors on the target cell tissues or organs are then inserted into these genes or free gene segments. Alternatively, it is possible to insert the gene sequences encoding corresponding epitopes into genetically unaltered parapoxviruses if this does not have any negative effects on viral replication or maturation and/or on the immunomodulatory properties of the viruses.

In addition, it is possible to use suitable immunological effective epitopes (e.g. HBV epitopes) to specifically reinforce the cell-mediated immune response against a pathogen.

For this, the organ-specifically, tissue-specifically and/or cell-specifically interacting/binding recombinant parapoxvirus ovis is additionally provided with specific epitopes, which are directed against one or more pathogens and which potentiate the immune response, and then employed in the relevant indication (for example against one or more of the abovementioned virus diseases such as chronic hepatitis B disease in humans). Alternatively, the gene sequences encoding appropriate epitopes can be inserted into genetically unaltered parapoxviruses if this does not have any negative effect on the replication or maturation of the virus and/or its immunomodulatory properties.

The recombinant parapoxvirus ovis is administered systemically (e.g. intramuscularly, subcutaneously, intraperitoneally or intravenously) or locally (e.g. into the relevant organ) in inactivated or non-inactivated form, depending on the clinical problem and/or the virus which is etiologically involved.

In this connection, the recombinant parapoxvirus is either present in lyophilized form, and then suspended in a suitable solvent immediately prior to administration, or else present in another suitable formulation.

In this connection, it may be necessary to give several administrations up to and including continuous infusion, in accordance with schedules which correspond to the requirements of the clinical problems.

Depending on the indication and/or the clinical problem, organ-specific, tissue-specific and/or cell-specific parapoxvirus ovis strains can be employed either as a monotherapy or in combination with biologically active low molecular weight compounds.

When parapoxvirus ovis is used in combination with biologically active low molecular weight compounds, the administration can take place either simultaneously or else staggered in time. Thus, it is possible, for example, initially to decrease or prevent viral replication using a low molecular weight compound (e.g. nucleotide analogs or other compounds) and then to bring about viral clearance using the recombinant parapoxvirus ovis. It is also possible to use such a combination therapy in the case of acute viral infections, for example.

### EXAMPLE

For Preparing and Testing a Targeting Mutant for the Herpesvirus Entry Mediator

Glycoprotein D (gD) of bovine herpesvirus 1 (BHV-1) is responsible for the binding of the virus to its target cell and for the penetration of the virus into the target cell, with other viral glycoproteins also being involved in this connection (Liang et al. 1991). Neutralizing gD-specific antibodies exert their function by interfering with the penetration of the virus, which is the step following adsorption of the virus (Okazaki et al. 1986). gD consequently serves as the viral site for binding the herpesvirus entry mediator (HVEM) (Montgomery et al. 1996). Cells which do not possess this herpesvirus entry mediator are resistant to infection with a variety of herpesviruses, for example human herpesvirus 1 [HSV-1] or BHV-1. Different BHV-1 strains, whose ability to express gD varies, also vary in their ability to penetrate the cells, with this ability being positively correlated with the content of gD (Fehler, 1991). Recombinant parapoxvirus ovis which carries gD on its surface can be used for targeting these HVEM binding sites on cells which express HVEM (e.g. MDBK cells). If these cells are infected with BHV-1, recombinant parapoxvirus which is expressing gD, or wild-type parapoxvirus, it should then be possible to measure the targeting of the HVEM by way of the penetration rate. In this connection, it is expected that gD-recombinant parapoxvirus will penetrate into the cells about as rapidly as BHV-1 and in any case more rapidly than wild-type parapoxvirus ovis, which is also able to infect MDBK cells.

Preparing the LacZ Mutant:

The vegf genes, which are present in duplicate in the genome, were deleted virtually completely from parapoxvirus ovis (strain D 1701), and an E. coli lacZ-xgpt expression cassette was in each case inserted at the sites (Rziha et al. 1999).

Preparing the Transfection Plasmid for the Homologous Recombination:

The BHV1 gD gene, including its signal sequence and membrane anchor (Tikoo et al. 1990), was amplified by PCR and blunt end-cloned into the EcoRV site of the vector pDVRec (Rziha et al. 1999). The congruence of the gD sequence in pDVRec with the original sequence was confirmed by sequencing (MWG Biotech).

Transfection:

The parapoxvirus lacZ mutant (D1701-RV) was transfected using the isolated plasmid pDVRec/gD and BKKL3A cells. The transfection was carried out using a 70 to 80% monolayer of the cells (6-well plate: cell number of approx. 4×10⁵ per well). The transfection reagent employed was the liposomal transfection reagent DOSPER. The cells were infected with the parapoxvirus lacZ mutant at an MOI of 0.1. 2 μg of plasmid DNA were mixed with DOSPER in a ratio of 1:3 and 1:4 and added to the cells following infection with the virus.

4 to 7 days after the transfection, the cells displayed a virus-specific cytopathic effect, and the virus was harvested by freezing and storing three times.

Plaque Purification:

BKKL3A cells were infected with the recombinant virus, which had been diluted in 10-fold steps (10⁻² to 10⁻⁶). The wells in which it was possible to see approx. 10 to 30 nascent virus plaques after a few days were overlaid with 300 μg/ml agarose Bluo-Gal (GIBCO). After the plates had been incubated at 37° C. for from 24 to 48 h (5% CO2), the white plaques were then picked. The virus material from the punched-out agarose block was eluted into medium overnight and the virus was multiplied once again (1st plaque purification). After the first plaque purification, the clones were hybridized with a P³²-labeled gD DNA probe in a dot blot. The positive recombinants were purified by means of at least three plaque purification steps.

Penetration Assay:

Bovine kidney cells (MDBK, ATCC No. CCL-22) were cultured in accordance with the ATCC instructions and were confluent at the beginning of the experiment. The cells were preincubated at 4° C. for 5 minutes. The medium was subsequently aspirated off and precooled (4° C.) a) BHV-1, b) gD-recombinant parapoxvirus or c) wild-type parapoxvirus ovis was added to the cells (MOI, 0.01). After that, the cells were incubated at 4° C. for 15 min, after which the medium was aspirated off and the cells were washed 1× with cold (4° C.) PBS. Subsequently, the incubation of the cells was continued in warm medium at a temperature of 37° C. in an incubator. After 10, 20, 30, 60 and 120 minutes, in each case 1 well was washed for approx. 45 seconds with citrate buffer while in each case 1 control well was washed with PBS. This thereby inactivated the viruses which had not up to that point penetrated into the cells. This gave the kinetics of the penetration. After the acid treatments, the cells were overlaid with medium (which contained 0.5% methylcellulose). After 3 days, the cells were fixed and stained and the plaques were determined in a plaque viewer using a scale of from 0 to ++++.

Result:

It was found that more than half of the viruses had penetrated the cytoplasmic membrane after only approx. 7 minutes in the case of BHV-1 and gD-recombinant parapoxvirus ovis (gDPPVO), whereas the wild-type parapoxvirus ovis (wt PPVO) required approx. 20 minutes to achieve this. These differences are significant (variance analysis together with post hoc comparison). This thereby demonstrated that a) it is possible to express a protein on the surface of parapoxvirus ovis and that b) this protein can be used for targeting specific receptors which are in turn expressed on particular cells and/or particular tissues.
1. Guidotti et al. (1996): Viral cross talk: Intracellular inactivation of the hepatitis B virus during an unrelated viral infection of the liver
2. Guidotti et al. (1994): Cytotoxic T lymphocytes inhibit hepatitis B virus gene expression by a noncytolytic mechanism in transgenic mice
3. Steinmassl, G., G. Wolf (1990): Bildung von Interleukin 2 und Interferon-γ durch mononukleare Leukozyten des Schweines nach in vitro-Stimulation mit verschiedenen Viruspräparaten (Formation of interleukin 2 and interferon-γ by pig mononuclear leucocytes following in-vitro stimulation with different virus preparations). J.Vet.Med.B37,5,321-331
4. Robinson, A. J. and Lyttle, D. J. (1992): Parapoxviruses: their biology and potential as recombinant vaccines. In: Recombinant Poxviruses, Chapter 9, 306-317 eds. M. Binns and G. Smith CRC Press, Boca Raton und WO 97/37031
5. Ishikawa, T. and Ganem, D. (1995): The pre-S domain of the large viral envelope protein determines host range in avian hepatitis B viruses. Proc. Natl.Acad. Sci. USA, 92 (14):6259-6263
6. Harris, J. D. and Lemoine, N. R. (1996): Strategies for targeted gene therapy. Trends in Genetics 12 (10): 400-405
7. Rensen, P. C. N., de Vrueh, L. A. and van Berkel, T. J. C.(1996): Targeting Hepatitis B Therapy to the Liver. Clin. Pharmacokinet. 31 (2)131-155
8. Barry, B. A., Dower, W. J. and Johnston, S. A. (1996): Toward cell-targeting gene therapy vectors: Selection of cell-binding peptides from random peptide-presenting phage libraries. Nature Medicine 2 (3):299-305
9. Kern, F., Surel, I. P., Brock, C., Freistedt, B., Radtke, H., Scheffold, A., Blasczyk, R., Reinke, P., Schneider-Mergener, J., Radbruch, A., Walden, P., Volk, H. D. (1998): T-cell epitope mapping by flow cytometry. Nat. Med. 4 (8): 975-978
10. P. Lucin, S. Jonjic, M. Messerle, B. Polic, H. Hengel, U. H. Koszinowski (1994): Late-Phase inhibition of murine cytomegalovirus replication by synergistic action of interferon gamma and tumor necrosis factor alpha. J. Gen. Virol 75:101-110; P.M.
11. Smith, R. M. Wolcott, R. Chervenak, S. R. Jennings (1994): Control of acute cutaneous herpes-simplex virus-Infection—T-cell mediated viral clearance is dependent upon interferon gamma. Virology 202 (1):76-88]
12. Y. Kawanashi, N. Hayashi, K. Katayama, K. ueda, T. Takehara, E. Miyoshi, E. Mita, A. Kasahara, H. Fusamoto, T. Kamada (1995): Tumor necrosis factor alpha and interferon gamma inhibit synergistically viral replication in hepatitis B virus replicating cells. J. Medical Virology 47 (3):272-277
13. Dhawan, S., L. M. Wahl, A. Heredia, Y. H. Zhang, J. S. Epstein, M. S. Meltzer, I. K. Hewlett (1995): Interferon gamma inhibits HIV-induced invasiveness of Monocytes. J. Leukocyte Biology, 58 (6):713-716
14. J. F. Bromberg, C. M. Horvath, Z. L. Wen, R. D. Schreiber, J. E. Darnell (1996): Transcriptionally active stat1 is required for the antiproliferative effects of both interferon alpha and interferon gamma. PNAS 93(15):7673-7678;
15. M. Klouche, H. Kirchner, F. Holzel (1994): Antiproliferative effects of inter-feron gamma in combination with alpha-difluoromethylornithine on human carcinoma cell cultures. J.Cancer Research and Clinical Oncology 120(12):706]
16. Fehler F (1991): Glycoprotein IV des bovinen Herpesvirus 1: funktionelle und strukturelle Eigenschaften eines essentiellen herpesviralen Glycoproteins (Glycoprotein IV of bovine herpesvirus I: functional and structural properties of an essential herpesvirus glycoprotein), Dissertation, Eberhard-Karls-University, Tübingen
17. Liang X, Babiuk L A, van Drunen, Little, Van den Hurk S, Fitzpatrick D R, Zamö T J (1991): Bovine herpesvirus 1 attachment to permissive cells is mediated by ist major glycoproteins gI, gIII and gIV. J. Virol. 65:1124-1132.
18. Montgomery R I, Warner M W, Lum B J, Spear P G (1996) Cell 87:427
19. Okazaki K, Honda E, Minetoma T, Kumagai T (1986): Mechanisms of neutralization by monoclonal antibodies to different antigenic sites on the bovine herpesvirus type 1 glycoproteins. Virology 150:260-264
20. Rziha H-J, Henkel M, Cottone R, Meyer M, Dehio C, Büttner M (1999): Parapoxviruses:potential alternative vectors for directing the immune response in permissive and non-permissive hosts. Journal of Biotechnology, Vol. 73, 235-242
21. Rziha H-J, Henkel M, Cottone R, Bauer B, Auge U, götz F, Pfaff E, Büttner M (1999): Generation of recombinant parapoxviruses: Non-essential genes suitable for foreign gene expression. Journal of Biotechnology (1.09.99)??
22. Tikoo S K, Fitzpatrick D R, Babiuk I A, Zamb T J (1990): Molecular Cloning, Sequencing, and Expression of functional bovine Herpesvirus 1 Glycoprotein gIV in Transfected Bovine Cells. Journal of Virology, Vol. 64, No.10, 5132-5142

1. The use of recombinant parapoxvirus possessing targeting properties for producing pharmaceuticals.

2. A pharmaceutical which comprises recombinant parapoxvirus possessing targeting properties.
